(19) United States
(12) Patent Application Publication
(10) Pub. No.: US 2024/0050338 A1
(43) Pub. Date: Feb. 15, 2024

(54) AQUEOUS EMULSION

(71) Applicant: L V M H RECHERCHE, Saint Jean De Braye (FR)

(72) Inventors: Masahiro KIKUCHI, Chiyoda-ku, Tokyo (JP); Mai OZAWA, Chiyoda-ku, Tokyo (JP); Takayoshi SAKODA, Chiyoda-ku, Tokyo (JP)

(73) Assignee: L V M H RECHERCHE, Saint Jean De Braye (FR)

(21) Appl. No.: 18/268,462

(22) PCT Filed: Dec. 22, 2020
(86) PCT No.: PCT/IB2020/001132

§ 371 Date: Jun. 20, 2023

(51) Int. Cl. A61K 8/34; A61K 8/73; A61K 8/37; A61K 8/06; A61Q 1/00; A61Q 19/00

(52) U.S. Cl. CPC A61K 8/345; A61K 8/731; A61K 8/375; A61K 8/062; A61Q 1/00; A61Q 19/00; A61K 2800/48

(57) ABSTRACT

The present invention relates to an aqueous emulsion comprising: (A) an ester of a fatty acid of 16 to 22 carbon atoms and polyglycerol, an ester of a fatty acid of 16 to 22 carbon atoms and glycerin and a monohydric alcohol of 16 to 22 carbon atoms; (B) a compound having at least two alcoholic hydroxyl groups and an alkyl group of 16 to 22 carbon atoms and no phosphorus atom, or a salt thereof, wherein component (B) is different from component (A); and (C) a cellulosic thickener and/or a polysaccharide thickener comprising glucose, glucuronic acid and rhamnose as constituent monosaccharides of the main chain.

### TECHNICAL FIELD

The present invention relates to an aqueous emulsion, in particular an oil-in-water type emulsion.

### BACKGROUND ART

Cosmetics with lamellar structures provide consumers with the sensation of a reinforced skin barrier. U.S. patent Ser. No. 10/449,126, for example, discloses an oil-in-water emulsion type cosmetic comprising a lamellar liquid crystal structure stabilized at low pH (3 to 4.8) by hydrophilic gelling agent, lipophilic surfactant (HLB 2 to 5), hydrophilic surfactant (HLB 8 to 12) and metal salt of monocetyl phosphate. But this composition does not deliver good spreadability, and/or may provide stickiness.

### Technical Problem

In addition, emulsion formulations with such lamellar structures, however, tend to easily lose their stability at high temperature, such as 50° C.

It is an object of the present invention to provide an aqueous emulsion with excellent stability at high temperature.

### Solution to Problem and Summary of Invention

The present invention relates to an aqueous emulsion comprising (A) an ester of a fatty acid of 16 to 22 carbon atoms and polyglycerol, an ester of a fatty acid of 16 to 22 carbon atoms and glycerin, and a monohydric alcohol of 16 to 22 carbon atoms, (B) a compound having at least two alcoholic hydroxyl groups and an alkyl group of 16 to 22 carbon atoms and no phosphorus atoms, or a salt thereof, which is different from component (A), and (C) a cellulosic thickener, and/or a polysaccharide thickener comprising glucose, glucuronic acid and rhamnose as constituent monosaccharides of the main chain.

In this aqueous emulsion, component (B) is preferably an alkyl glyceryl ether, or an organic acid monoglyceride and/or its salt.

In this aqueous emulsion, component (B) is also preferably batyl alcohol, or glyceryl stearate citrate and/or its salt.

The cellulosic thickener in the aqueous emulsion preferably comprises an alkyl group of 10 to 30 carbon atoms.

The cellulosic thickener in the aqueous emulsion also preferably comprises hydroxypropyl methyl cellulose stearoxy ether.

The polysaccharide thickener in the aqueous emulsion may further comprise rhamnose or fucose as a constituent monosaccharide of a side chain.

The aqueous emulsion may be an external preparation for skin.

According to the invention there is provided an aqueous emulsion with excellent stability at high temperature, in particular 50° C. for 1 month or 45° C. for 3 months. The present invention also relates to a cosmetic process for caring and/or making-up keratinic materials, comprising the application onto the keratinic materials, in particular the skin, of the aqueous emulsion of the invention.

### DETAILED DESCRIPTION OF THE INVENTION

A preferred embodiment of the invention will now be described in detail. However, the present invention is not limited to the embodiment described below.

The aqueous emulsion of the invention comprises (A) an ester of a fatty acid of 16 to 22 carbon atoms and polyglycerol, an ester of a fatty acid of 16 to 22 carbon atoms and glycerin, and a monohydric alcohol of 16 to 22 carbon atoms, (B) a compound having at least two alcoholic hydroxyl groups and an alkyl group of 16 to 22 carbon atoms and no phosphorus atoms, or a salt thereof, which is different from component (A), and (C) a cellulosic thickener, and/or a polysaccharide thickener comprising glucose, glucuronic acid and rhamnose as constituent monosaccharides of the main chain.

The aqueous emulsion of this embodiment not only has excellent stability at high temperature, but is also advantageous by having excellent spreadability and reduced stickiness on the skin.

The aqueous emulsion of the embodiment may comprise a lamellar structure. A lamellar structure is a structure in which an amphiphilic substance such as a surfactant, is oriented with oil and water interfaces stacked in a manner to form alternating oil phase and aqueous phase layers. The lamellar structure may also be one derived from components (A), (B) and (C), and the other components mentioned below, for example. A lamellar structure is expected to improve skin barrier function. Since the aqueous emulsion of the embodiment comprises components (A), (B) and (C), it can stably comprise a lamellar structure for long periods even at a high temperature of 45 to 50° C., for example.

Component (A)

The aqueous emulsion of this embodiment comprises, as component (A), a combination of an ester of a fatty acid of 16 to 22 carbon atoms and polyglycerol, an ester of a fatty acid and 16 to 22 carbon atoms and glycerin, and a monohydric alcohol of 16 to 22 carbon atoms. Component (A) in the aqueous emulsion of the embodiment functions as an emulsifier.

In a particular embodiment, in the ester of a fatty acid of 16 to 22 carbon atoms and polyglycerol (hereunder also referred to as “component (A1)”), the number of carbon atoms of the fatty acid is preferably 16 to 20 and more preferably 18. Examples for constituent fatty acids of component (A1) include palmitic acid, palmitoleic acid, stearic acid, oleic acid, linoleic acid, α-linolenic acid, γ-linolenic acid, isostearic acid, arachidic acid, arachidonic acid and behenic acid. The fatty acid may be saturated or unsaturated, but it is preferably saturated.

The polymerization degree of the glycerin in component (A1) may be 2 to 10, preferably 2 to 4 and more preferably 2.

In a particular embodiment, component (A1) is selected in the group consisting of polyglyceryl-2 stearate, polyglyceryl isostearate-2, is polyglyceryl oleate-2, and mixtures thereof for example.

In a particular embodiment, the total content of component(s) (A1) in the total of component (A) ranges from 30 to 60 mass %, in particular 35 to 50 mass % or 40 to 50 mass %, for example.

In a particular embodiment, in the ester of a fatty acid of 16 to 22 carbon atoms and glycerin (hereunder also referred to as “component (A2)”), the number of carbon atoms of the fatty acid is preferably 16 to 20 and more preferably 18. Examples for constituent fatty acids of component (A2) include palmitic acid, palmitoleic acid, stearic acid, oleic acid, linoleic acid, α-linolenic acid, γ-linolenic acid, arachidic acid, arachidonic acid and behenic acid. The fatty acid may be saturated or unsaturated, but it is preferably saturated. In a particular embodiment, component (A2) is selected in the group consisting of glyceryl stearate, glyceryl oleate, glyceryl myristate, glyceryl palm fatty acids, glyceryl undecylenic acid, and mixtures thereof, for example.

In a particular embodiment, the total content of component(s) (A2) in the total of component (A) ranges from 10 to 50 mass %, in particular 20 to 40 mass % or 25 to 35 mass %, for example.

In a particular embodiment, in the ester of a monohydric alcohol of 16 to 22 carbon atoms (hereunder also referred to as “component (A3)”), the number of carbon atoms is preferably 16 to 20 and more preferably 18. Examples for component (A3) include myristyl alcohol, cetanol, stearyl alcohol, isostearyl alcohol, oleyl alcohol, arachidyl alcohol, and behenyl alcohol. The monohydric alcohol may be saturated or unsaturated, but it is preferably saturated. In a particular embodiment, component (A3) is preferably stearyl alcohol oleyl alcohol, cetanol, behenyl alcohol, or mixture thereof.

In a particular embodiment, the total content of component(s) (A3) in the total of component (A) may be 10 to 40 mass %, 15 to 35 mass % or 20 to 30 mass %, for example.

In a particular embodiment, the total content of component (A) with respect to the total amount of the aqueous emulsion may be 0.5 mass % or greater, 1 mass % or greater, 1.2 mass % or greater, 1.5 mass % or greater, 1.8 mass % or greater, 2.0 mass % or greater, 2.5 mass % or greater, 3.0 mass % or greater, 3.5 mass % or greater or 3.8 mass % or greater, and 10 mass % or lower, 8 mass % or lower, 6 mass % or lower, 5 mass % or lower, 4 mass % or lower, 3 mass % or lower, 2.5 mass % or lower, 2.2 mass % or lower or 2 mass % or lower, for example. In particular, the total content of component (A) ranges from 0.5 mass % to 10 mass %, in particular 2 mass % to 8 mass %, preferably 2.5 mass % to 6 mass % with respect to the total amount of the aqueous emulsion.

Component (A) is preferably a solid at ordinary temperature (25° C., for example). In a particular and preferred embodiment, component (A) is a combination of polyglyceryl-2 stearate, glyceryl stearate and stearyl alcohol. PolyAquol 2w (Innovacos Corp.) is a suitable example of a commercial product that comprises a combination of polyglyceryl-2 stearate, glyceryl stearate and stearyl alcohol.

Component (B)

The aqueous emulsion of the invention comprises, as component (B), a compound having at least two alcoholic hydroxyl groups and an alkyl group of 16 to 22 carbon atoms and no phosphorus atoms, or a salt thereof Component (B) is different from any of the compounds of component (A). Component (B) is an amphiphilic substance having a hydrophilic

portion and a lipophilic portion in the molecule of the compound. If the aqueous emulsion of this embodiment includes component (B) having the specified structure in combination with component (A) and component (C), then it will be easier to obtain high viscosity at room temperature due to reinforcement of the lamellar structure, and phase separation of the emulsion can be inhibited even after passage of time.

In a particular embodiment, the alkyl group of the compound of component (B) is one of 16 to 20 carbon atoms or 16 to 18 carbon atoms. Component (B) may have 2, 3, 4, 5 or 6 alcoholic hydroxyl groups in one molecule, for example.

Component (B) is a compound having no phosphorus atoms. The present inventors have found that rapid phase separation can be produced in the emulsion when it is obtained using a monoalkyl phosphate as an amphiphilic wax. Component (B) is preferably a compound consisting of a carbon atom, a hydrogen atom and an oxygen atom, or its salt.

in a particular embodiment, component (B) is an alkyl glyceryl ether, or an organic acid monoglyceride and/or its salt, for example. An alkyl glyceryl ether may have two alcoholic hydroxyl groups, for example. Batyl alcohol, chimyl alcohol, and selachyl alcohol are examples for an alkyl glyceryl ether having at least two alcoholic hydroxyl groups and an alkyl group of 16 to 22 carbon atoms, and having no phosphorus atoms.

An organic acid monoglyceride may be a monoester of an organic acid with an ester of a fatty acid of 16 to 22 carbon atoms and glycerin, for example. The organic acid in an organic acid monoglyceride may be an aliphatic hydroxy acid such as citric acid. In a particular embodiment, glyceryl stearate citrate, glyceryl fatty acid succinate, or a salt thereof, are examples for an organic acid monoglyceride or its salt having at least two alcoholic hydroxyl groups and an alkyl group of 16 to 22 carbon atoms, and having no phosphorus atoms. Component (B) may be used as a single type alone, or multiple types may be used in combination.

In the aqueous emulsion of this embodiment, the total content of component (B) with respect to the total amount of the aqueous emulsion may be 0.1 mass % or greater, 0.2 mass % or greater, 0.5 mass % or greater, 0.8 mass % or greater, 1.0 mass % or greater or 1.2 mass % or greater, and 3.0 mass % or lower, 2.5 mass % or lower, 2.2 mass % or lower, 2.0 mass % or lower, 1.8 mass % or lower, 1.7 mass % or lower, 1.6 mass % or lower, 1.4 mass % or lower, 1.2 mass % or lower or 1.0 mass % or lower, for example. In particular, the total content of component (B) ranges from 0.1 mass % to 3 mass %, in particular from 0.2 mass % to 2 mass % with respect to the total amount of the aqueous emulsion.

Component (C)

The aqueous emulsion of the invention comprises, as component (C), a cellulosic thickener and/or a polysaccharide thickener that comprises glucose, glucuronic acid and rhamnose as constituent monosaccharides of the main chain. If the aqueous emulsion comprises these thickeners, it will be able to exhibit high stability at high temperature while maintaining suitable viscosity.

Examples of cellulosic thickeners include ethyl cellulose, hydroxyethyl cellulose, hydroxypropyl cellulose, hydroxypropyl methyl cellulose, hydroxypropyl methyl cellulose stearoxy ether, cellulose gum, is carboxymethyl cellulose, and mixtures thereof In a particular embodiment, the cellulosic thickener preferably comprises an alkyl group of 10 to 30 carbon atoms from the viewpoint of further increasing the high temperature stability and further inhibiting stickiness of the aqueous emulsion. In a particular and preferred embodiment, hydroxypropyl methyl cellulose stearoxy ether is an example of a cellulosic thickener with an alkyl group of 10 to 30 carbon atoms.

The polysaccharide thickener may have a main chain with a structural unit comprising glucose, glucuronic acid, glucose and rhamnose bonded linearily in that order. The polysaccharide thickener may also have a side chain as part of the polysaccharide structure, or it may lack a side chain.

The side chain may have one or more selected from the group consisting of fucose, glucose, rhamnose and mannose as a constituent monosaccharide. The polysaccharide thickener may be obtained from a microbe such as a bacterium. In a particular embodiment, the polysaccharide thickener is welan gum, gellan gum, diutan gum, or mixtures thereof, for example. The polysaccharide thickener may have a main chain with a structural unit comprising glucose, glucuronic acid, glucose and rhamnose bonded linearily, with fucose as a side chain.

Component (C) may be used as a single type alone, or multiple types may be used in combination. The total content of component (C) with respect to the total amount of the aqueous emulsion may be 0.01 mass % or greater, 0.02 mass % or greater, 0.03 mass % or greater, 0.05 mass % or greater, 0.1 mass % or greater, 0.2 mass % or greater or 0.3 mass % or greater, and 1.0 mass % or lower, 0.8 mass % or lower, 0.6 mass % or lower, 0.5 mass % or lower, 0.4 mass % or lower, 0.3 mass % or lower, 0.2 mass % or lower, 0.15 is mass % or lower or 0.1 mass % or lower, for example. In particular, the total content of component (C) ranges from 0.01 mass % to 1.0 mass %, in particular from 0.05 mass % to 0.5 mass % with respect to the total amount of the aqueous emulsion. The aqueous emulsion of this embodiment has high temperature stability even with a low amount of thickener used.

In a particular embodiment, the aqueous emulsion also comprises a thickener other than component (C). Examples of thickeners other than component (C) include polysaccharide thickeners such as xanthan gum, alkyl-modified carboxyvinyl polymers such as acrylates/c10-30 alkyl acrylate crosspolymer, carbomer, a polymer containing (meth) acryloylalkyltaurine or a salt thereof as a repeating unit.

The content of the thickener other than component (C) with respect to the total amount of the aqueous emulsion may be 0.01 mass % or greater, 0.1 mass % or greater, 0.15 mass % or greater, 0.2 mass % or greater, 0.3 mass % or greater, 0.4 mass % or greater or 0.5 mass % or greater, and 1.0 mass % or lower, 0.8 mass % or lower, 0.5 mass % or lower, 0.3 mass % or lower or 0.2 mass % or lower, for example. In particular, the content of the thickener other than component (C) ranges from 0.01 mass % to 1 mass %, with respect to the total amount of the aqueous emulsion.

The total content of component (C) and a thickener other than component (C) with respect to the total amount of the aqueous emulsion may be 0.01 mass % or greater, 0.02 mass % or greater, 0.03 mass % or greater, 0.05 mass % or greater, 0.1 mass % or greater, 0.2 mass % or greater or 0.3 mass % or greater, and 1.5 mass % or lower, 1.0 mass % or lower, 0.8 mass % or lower, 0.6 mass % or lower, 0.5 mass % or lower, 0.4 mass % or lower, 0.3 mass % or lower, 0.2 mass % or lower, 0.15 mass % or lower or 0.1 mass % or lower, for example. In particular, the total content of component (C) and a thickener other than component (C) ranges from 0.01 mass % to 1.5 mass % with respect to the total amount of the aqueous emulsion.

Oil Agent

In a particular embodiment, the aqueous emulsion of the invention also comprises an oil agent other than components (A), (B) and (C). The oil agent may be a liquid oil or a paste oil, for example. A liquid oil may be either a non-polar liquid oil or a polar liquid oil. Examples of liquid oils include coco-caprylate/caprate, squalane, sunflower oil, dimethicone, meadow foam oil, almond oil, polyglyceryl-2 triisostearate, caprylic/capric triglyceride, isotridecyl isononanoate, isononyl isononanoate, ethylhexyl palmitate, pentaerythrityl tetraethylhexanoate, pentaerythrityl tetraisostearate, cetyl ethylhexanoate, hydrogenated polydecene, hydrogenated polyisobutene, and mixtures thereof.

The content of the oil agent with respect to the total amount of the aqueous emulsion may be 5 mass % or greater, 7 mass % or greater, 8 mass % or greater, 10 mass % or greater, 12 mass % or greater, 14 mass % or greater or 16 mass % or greater, and 40 mass % or lower, 30 mass % or lower, 20 mass % or lower, 18 mass % or lower, 17 mass % or lower, 15 mass % or lower, 13 mass % or lower or 10 mass % or lower, for example. In particular, the content of the oil agent ranges from 5 mass % to 40 mass %, in particular from 10 mass % to 30 mass % with respect to the total amount of the aqueous emulsion.

The content of the liquid oil with respect to the total amount of the aqueous emulsion may be 3 mass % or greater, 5 mass % or greater, 7 mass % or greater, 8 mass % or greater, 10 mass % or greater, 12 mass % or greater or 14 mass % or greater, and 40 mass % or lower, 30 mass % or lower, 20 mass % or lower, 18 mass % or lower, 17 mass % or lower, 15 mass % or lower, 13 mass % or lower, 10 mass % or lower or 8 mass % or lower, for example. In particular, the content of the liquid oil ranges from 3 mass % to 40 mass %, in particular from 10 mass % to 30 mass % with respect to the total amount of the aqueous emulsion.

In a particular embodiment, the aqueous emulsion also comprises additives commonly used in cosmetics, such as a humectant, a pH adjustor, a fragrance, a preservative, an active component, an antioxidant, a UV blocking agent, a whitening agent, a pigment, a colorant, an anti-inflammatory active, an anti-acne active, or a skin conditioning agent. In a particular embodiment, the aqueous emulsion comprises an active component, in particular a whitening agent.

Examples of humectants include polyhydric alcohols of 3 to 5 carbon atoms. Examples of polyhydric alcohols include those with 2 or 3 alcoholic hydroxyl groups. Examples of polyhydric alcohols of 3 to 5 carbon atoms include glycerin, 1,3-butylene glycol, pentylene glycol and propanediol.

The content of a polyhydric alcohol of 3 to 5 carbon atoms with respect to the total amount of the aqueous emulsion may be 2 mass % or greater, 5 mass % or greater, 8 mass % or greater, 10 mass % or greater, 12 mass % or greater, 15 mass % or greater, 18 mass % or greater or 20 mass % or greater, and 30 mass % or lower, 28 mass % or lower, 25 mass % or lower, 23 mass % or lower, 20 mass % or lower, 18 mass % or lower or 15 mass % or lower, for example. In particular, the content of a polyhydric alcohol of 3 to 5 carbon atoms ranges from 2 mass % to 30 mass %, in particular from 10 mass % to 30 mass % with respect to the total amount of the aqueous emulsion.

The pH of the aqueous emulsion of this embodiment may be 3 to 9 or 4 to 8, for example.

In a particular embodiment, the aqueous emulsion of the invention has a viscosity ranging from 1,000 to 100,000 mPa·s or from 3,000 to 50,000 mPa·s, for example, when measured at 25° C. The viscosity can be measured by measuring the shear viscosity using a rotational viscometer (for example, a Rheolab QC by Anton Paar GmbH, spindle; ST-24-2D/2V/2V-30, a rotational speed of 50 rpm).

In a particular embodiment, the aqueous emulsion of the invention is an oil-in-water type emulsion. The aqueous emulsion of the invention also contains water. The total content of water with respect to the total amount of the aqueous emulsion may be 40 mass % or greater, 43 mass % or greater, 45 mass % or greater, 47 mass % or greater, 49 mass % or greater, 51 mass % or greater, 53 mass % or greater, 55 mass % or greater, 58 mass % or greater, 60 mass % or greater or 61 mass % or greater, and 70 mass % or lower, 65 mass % or lower, 63 mass % or lower, 61 mass % or lower, 59 mass % or lower or 57 mass % or lower, for example. In particular, the total content of water ranges from 40 mass % to 70 mass %, in particular from 49 mass % to 65 mass % with respect to the total amount of the aqueous emulsion.

In a particular embodiment, the aqueous emulsion of the invention is produced, for example, by stirring and mixing component (A), component (B) and component (C), with the other thickener, oil agent and/or emulsifier, while heating at 80 to 85° C., and then cooling the mixture to room temperature and mixing in the other components.

In a particular embodiment, the aqueous emulsion of the invention is in the form of a cream or a fluid emulsion, for example. The aqueous emulsion of the embodiment can exhibit high viscosity at room temperature when the emulsion is in the form of a cream, for example, since the emulsion can comprise a stable lamellar structure. The aqueous emulsion is preferably a skin care cosmetic.

### EXAMPLES

Aqueous emulsions were prepared with the mixing ratios listed in Tables 1 and 2. The numerical values in Tables 1 and 2 are percentages. Their totals are all 100%. First, a liquid oil other than dimethicone, a paste oil, an emulsifier and a co-emulsifier were dissolved at 85° C. Separately, an active component and a pH adjustor were dissolved in at room temperature. A humectant, a preservative and citric acid were then loaded into the tank and mixed in at 80 to 85° C. Thickeners were added to and mixed in the tank. The dissolved liquid oil other than dimethicone, the paste oil, the emulsifier and the co-emulsifier were then added to and mixed in the tank. Dimethicone was further added to the tank and the mixture was emulsified for 10 minutes. The mixture was then cooled to 30° C., mixing with the other materials at the point where the temperature fell below 40° C., to obtain an aqueous emulsion.

TABLE 1

| Example No. | | 1 | 2 | 3 | 4 | 5 | 6 | 7 |
|---|---|---|---|---|---|---|---|---|
| Liquid oil | Coco-caprylate/caprate | 9.0 | 9.0 | 9.0 | 9.0 | 9.0 | 2.5 | 2.5 |
| | Squalane | 6.0 | 6.0 | 6.0 | 6.0 | 6.0 | 3.0 | 3.0 |
| | Hybrid sunflower oil | — | — | — | — | — | 2.0 | 2.0 |
| | Dimethicone | 0.4 | 0.5 | 0.5 | — | 0.5 | 0.4 | 0.4 |
| Paste oil | Shea butter and citric | 1.0 | 1.0 | 1.0 | 1.0 | 1.0 | 0.5 | 0.5 |
| | acid 0.001% | | | | | | | |
| (A) | Polyglyceryl-2 stearate, | 4.0 | 4.0 | 4.0 | 4.0 | 4.0 | 2.0 | 2.0 |
| | glyceryl stearate, stearyl | | | | | | | |
| | alcohol | | | | | | | |
| | Behenyl alcohol | 0.4 | — | — | — | — | — | — |
| (B) | Batyl alcohol | — | — | 0.8 | — | — | 0.2 | 0.2 |
| | Glyceryl stearate citrate | 1.6 | 0.8 | — | 0.8 | 0.8 | — | — |
| Humectant | Glycerin | 10.0 | 10.0 | 10.0 | 10.0 | 10.0 | — | — |
| | 1,3-Butylene glycol | 4.0 | 4.0 | 4.0 | 4.0 | 4.0 | 4.0 | 4.0 |
| | Pentylene glycol | 2.4 | 2.4 | 2.4 | 2.4 | 2.4 | 2.4 | 2.4 |
| | Propanediol | 5.6 | 5.6 | 5.6 | 5.6 | 5.6 | 5.6 | 5.6 |
| Antiseptic | Chlorphenesin | 0.3 | 0.3 | 0.3 | 0.3 | 0.3 | 0.3 | 0.3 |
| agent | | | | | | | | |
| (C) | Hydroxypropyl methyl | 0.4 | 0.4 | 0.4 | 0.4 | — | — | — |
| | cellulose stearoxy ether, | | | | | | | |
| | cyclodextrin | | | | | | | |
| | Hydroxypropyl methyl | — | — | — | — | 0.3 | 0.1 | — |
| | cellulose stearoxy ether | | | | | | | |
| | Polysaccharide 1 | — | — | — | — | — | — | 0.1 |
| Thickener | Acrylates/C10-30 alkyl | — | — | — | — | — | 0.1 | 0.2 |
| | acrylate crosspolymer | | | | | | | |
| | Xanthan gum 1 | 0.2 | 0.2 | 0.2 | — | 0.2 | 0.1 | 0.1 |
| Active | Gluconolactone | 0.5 | 0.5 | 0.5 | 0.5 | 0.5 | — | — |
| component | 3-o-Ethyl ascorbic acid | 1.0 | 1.0 | 1.0 | 1.0 | 1.0 | — | — |
| | Other active ingredients | — | — | — | — | — | 14.0 | 15.0 |
| pH adjustor | Citric acid | 0.1 | 0.1 | 0.1 | 0.1 | 0.1 | — | — |
| | Sodium hydroxide | — | 0.3 | 0.3 | 0.3 | 0.3 | — | — |
| | Tromethamine | 0.3 | — | — | — | — | 0.1 | 0.4 |

| | | | | | | | |
|---|---|---|---|---|---|---|---|
| Water | 52.7 | 53.8 | 53.8 | 53.4 | 53.8 | 62.7 | 61.3 |
| Fragrance | 0.2 | 0.2 | 0.2 | 0.2 | 0.2 | 0.2 | 0.2 |

TABLE 2

| | | | | 3 | | 5 | |
|---|---|---|---|---|---|---|---|
| | | 1 | 2 | Without | 4 | Without | 6 |
| | | Without | Without | (B) nor | Without | (B) nor | Without |
| Comparative Example No. | | (A) | 2 (C) | (C) | (C) | (C) | (C) |
| Liquid oil | Coco-caprylate/caprate | 9.0 | 14.0 | 14.0 | 9.0 | 9.0 | 9.0 |
| | Squalane | 6.0 | — | — | 6.0 | 6.0 | 6.0 |
| | Dimethicone | 0.4 | — | — | — | — | — |
| Paste oil | Shea butter and citric acid | 1.0 | 2.0 | 2.0 | 1.0 | 1.0 | 1.0 |
| | 0.001% | | | | | | |
| (A) | Polyglyceryl-2 stearate, | — | 4.0 | 4.0 | 4.0 | 4.0 | 4.0 |
| | glyceryl stearate, steary1 | | | | | | |
| | alcohol | | | | | | |
| | Glyceryl stearate | 1.2 | — | — | — | — | — |
| | Stearyl alcohol | 1.0 | — | — | — | — | — |
| | Behenyl alcohol | 0.4 | — | — | — | 0.8 | — |
| (B) | Glyceryl stearate citrate | 1.6 | 0.8 | — | 0.8 | — | 0.8 |
| Humectant | Glycerin | 10.0 | 10.0 | 10.0 | 10.0 | 10.0 | 10.0 |
| | 1,3-Butylene glycol | 4.0 | 4.0 | 4.0 | 4.0 | 4.0 | 4.0 |
| | Pentylene glycol | 2.4 | 2.4 | 2.4 | 2.4 | 2.4 | 2.4 |
| | Propanediol | 5.6 | 5.6 | 5.6 | 5.6 | 5.6 | 5.6 |
| Antiseptic | Chlorphenesin | 0.3 | 0.3 | 0.3 | 0.3 | 0.3 | 0.3 |
| agent | | | | | | | |
| (C) | Hydroxypropyl methyl | 0.4 | — | — | — | — | — |
| | cellulose stearoxy ether, | | | | | | |
| | cyclodextrin | | | | | | |
| Thickener | Xanthan gum 1 | 0.2 | — | — | — | — | 0.2 |
| | Xanthan gum 2 | — | — | — | — | — | 0.2 |
| | Sclerotium gum | — | — | — | — | — | 0.2 |
| Active | Gluconolactone | 0.5 | — | — | 0.5 | 0.5 | 0.5 |
| component | 3-o-Ethyl ascorbic acid | 1.0 | 1.0 | 1.0 | 1.0 | 1.0 | 1.0 |
| | Other active ingredients | — | — | — | — | — | — |
| pH adjustor | Citric acid | 0.1 | 0.2 | 0.2 | 0.1 | 0.1 | 0.1 |
| | Sodium hydroxide | — | 0.0 | 0.0 | 0.3 | 0.3 | 0.3 |
| | Tromethamine | 0.3 | — | — | — | — | — |

| | | | | | | |
|---|---|---|---|---|---|---|
| Water | 54.5 | 55.5 | 56.3 | 54.8 | 54.8 | 54.4 |
| Fragrance | 0.2 | 0.2 | 0.2 | 0.2 | 0.2 | 0.2 |

The following are the details for the materials listed in the tables.
Polyglyceryl-2 stearate, glyceryl stearate, stearyl alcohol: POLYAQUOL 2W, Innovacos Corp.
Batyl Alcohol: Nikkol Batyl Alcohol 100, Nikko Chemicals
Glyceryl stearate citrate: SUNSOFT GSC-C, Taiyo Kagaku Co., Ltd.
Hydroxypropyl methyl cellulose stearoxy ether, cyclodextrin: CELGELA TS9, Daido Chemical Industry Co., Ltd.
Hydroxypropyl methyl cellulose stearoxy ether: SANGELOSE 90L, Daido Chemical Industry Co., Ltd.
Polysaccharide 1: A microbial-derived polysaccharide thickener with the main chain as a structural unit consisting of glucose, glucuronic acid, glucose and rhamnose bonded linearily in that order, and with fucose as a side chain.
Acrylates/c10-30 alkyl acrylate crosspolymer: CARBOPOL ETD 2020, Lubrizol
Xanthan gum 1: RHODICARE XC, Solvey
Xanthan gum 2: RHODICARE T, Solvey

[Evaluation]

The stability of each obtained emulsion was evaluated by the following method. 30 g of the emulsion was placed in a transparent vessel (50 mL). The glass bottle was capped and sealed. The glass bottle was stored at constant temperature of 50° C. or 45° C. The presence of phase separation was visually observed after standing for one month at 50° C., and after standing for three months at 45° C. The results are shown in Tables 3 and 4. In the tables, “Good” means that no phase separation was observed, “Separate” means that oil phase/aqueous phase separation was observed, and “Not emulsified” means that emulsification did not take place during preparation.

TABLE 3

| Example No. | 1 | 2 | 3 | 4 | 5 | 6 | 7 |
|---|---|---|---|---|---|---|---|
| Stability at 50° C. | Good | Good | Good | Good | Good | Good | Good |
| for one month | | | | | | | |
| Stability at 45° C. | Good | Good | Good | Good | Good | Good | Good |
| for three months | | | | | | | |

TABLE 4

| Comparative Example No. | 1 | 2 | 3 | 4 | 5 | 6 |
|---|---|---|---|---|---|---|
| Stability at 50° C. for one month | Not | Good | Separate | Good | Separate | Separate |
| | emulsified | | | | | |
| Stability at 45° C. for three months | Not | Separate | Separate | Separate | Separate | Good |
| | emulsified | | | | | |

All of the aqueous emulsions obtained in the Examples exhibited excellent stability without phase separation after a period of one month at 50° C. or three months at 45° C. The mixture of Comparative Example 1, however, could not be emulsified. The aqueous emulsions obtained in the other Comparative Examples exhibited phase separation after either or both one month at 50° C. or three months at 45° C.

Upon confirming the textures of the prepared aqueous emulsions, all of the aqueous emulsions of the Examples were found to have low stickiness and excellent spreadability. The aqueous emulsion of Example 1 had particularly low stickiness and particularly excellent spreadability. The aqueous emulsion of Comparative Example 6, however, had poor spreadability. Comparative Examples 2, 3, and 6 produced a sticky feel when taken up with a finger. All these results demonstrate that the combination of components (A) to (C) as claimed in the present invention provides an aqueous emulsion with good stability even at high temperature (45° C.-50° C. for several months), and having low stickiness and particularly excellent spreadability.

1. An aqueous emulsion comprising:
(A) an ester of a fatty acid of 16 to 22 carbon atoms and polyglycerol, an ester of a fatty acid of 16 to 22 carbon atoms and glycerin and a monohydric alcohol of 16 to 22 carbon atoms;
(B) a compound having at least two alcoholic hydroxyl groups and an alkyl group of 16 to 22 carbon atoms and no phosphorus atom, or a salt thereof, wherein component (B) is different from component (A); and
(C) a cellulosic thickener and/or a polysaccharide thickener comprising glucose, glucuronic acid and rhamnose as constituent monosaccharides of the main chain.

2. The aqueous emulsion according to claim 1, wherein component (B) is an alkyl glyceryl ether, or an organic acid monoglyceride and/or its salt.

3. The aqueous emulsion according to claim 1, wherein component (B) is batyl alcohol and/or glyceryl stearate citrate and/or its salt.

4. The aqueous emulsion according to claim 1, wherein the cellulosic thickener comprises an alkyl group of 10 to 30 carbon atoms.

5. The aqueous emulsion according to claim 4, wherein the cellulosic thickener comprises hydroxypropyl methyl cellulose stearoxy ether.

6. The aqueous emulsion according to claim 1, wherein the polysaccharide thickener further comprises rhamnose or fucose as a constituent monosaccharide of a side chain.

7. The aqueous emulsion according to claim 1, wherein the aqueous emulsion is an external preparation for skin.

8. The aqueous emulsion according to claim 1, wherein the aqueous emulsion is in the form of a cream or a fluid emulsion.

9. A cosmetic process for caring for and/or making-up the keratinic materials, comprising the application onto the keratinic materials, of an aqueous emulsion as defined in claim 1.

10. The cosmetic process according to claim 9, wherein the aqueous emulsion is applied onto the skin.
